(19) United States
(12) Patent Application Publication
(10) Pub. No.: US 2016/0299118 A1
(43) Pub. Date: Oct. 13, 2016

(54) METHOD FOR ANALYSIS OF TRACE LEVELS OF CHEMICAL ADDITIVES IN OIL RECOVERY PRODUCTION FLUIDS

(71) Applicant: DOW GLOBALTECHNOLOGIES LLC, Midland, MI (US)

(72) Inventors: Jim C. LUONG, Sherwood Park (CA); Ronda L. GRAS, Edmonton (CA); William L. WINNIFORD, Lake Jackson, TX (US); Naoko AKIYA, Missouri City, TX (US)

(21) Appl. No.: 15/037,733

(22) PCT Filed: Nov. 19, 2014
(86) PCT No.: PCT/US2014/066267

§ 371 Date: May 19, 2016

Related U.S. Application Data
(60) Provisional application No. 61/916,371, filed on Dec. 16, 2013.

(51) Int. Cl. G01N 33/28; C09K 8/594; G01N 30/72; G01N 30/46; E21B 49/08; G01N 30/68

(52) U.S. Cl. CPC G01N 33/2823; E21B 49/086; G01N 30/68; G01N 30/7206; G01N 30/463; G01N 33/2835; C09K 8/594; E21B 43/2406

(57) ABSTRACT

Disclosed is a method for analyzing and/or monitoring trace levels of chemical additives in production fluids for oil recovery processes, specifically from heavy oil and/or oil sands. The method comprises and extraction step coupled with a multi-dimensional gas chromatography analysis.

### FIELD OF THE INVENTION

The present invention relates to monitoring trace levels of chemical additives; more specifically, monitoring glycol ether additives, in oil production fluids; more specifically from oil recovery processes for heavy oil and from oil sands.

### BACKGROUND OF THE INVENTION

There are many petroleum-bearing formations from which oil cannot be recovered by conventional means because the oil is so viscous that it will not flow from the formation to a conventional oil well. Examples of such formations are the bitumen deposits in Canada and in the United States and the heavy oil deposits in Canada, the United States, and Venezuela. In these deposits, the oil is so viscous under the prevailing temperatures and pressures within the formations that it flows very slowly (or not at all) in response to the force of gravity.

For oil sand deposits less than 70 meters deep, bitumen is recovered by mining the sands, then separating the bitumen from the reservoir rock by hot water processing, and finally upgrading the natural bitumen to synthetic crude oil. In deeper bitumen deposits, steam is injected into the reservoir in order to mobilize the oil for in situ production. Typical processes are steam-assisted gravity drainage (SAGD) and cyclic steam stimulation (CSS). The resulting product may be upgraded onsite or mixed with diluent and transported to an upgrading facility.

Chemical additives are used in different steps of the in situ bitumen production process. A chemical may be co-injected with steam with the aim of enhancing the production rate. Emulsion breakers, reverse emulsion breakers, and water clarifiers are typically used in the field to aid the oil-water separation and further de-oiling of water, to ensure that the bitumen stream meets the basic sediment and water (BS&W) specification and that the recycled water is sufficiently clean as the boiler feed water for steam generation. U.S. Pat. Nos. 5,045,212; 4,686,066; and 4,160,742 disclose examples of chemical demulsifiers used for breaking emulsions. Emulsion breakers are also used in mining operations to reduce the water content in the oil stream produced by hot water processing and subsequent treatment steps. These different chemical additives are typically trace levels dosed on a parts-per-million (ppm) level.

Effective evaluation of these chemicals requires monitoring the presence of the chemicals in the production streams. The chemical's presence needs to be established before any beneficial or adverse effects observed in a process can be attributed to the use of the chemical. Detection is particularly important for downhole chemicals, since there can be significant delay between the time the chemicals are injected and the time they return to the surface. However, detecting chemical additives in the field can be difficult and can require rigorous analytical techniques. Production streams, starting from produced emulsion at the well head and through different points along the oil-water separation and water treatment steps, are mixtures of bitumen and water with varying bitumen content. Since bitumen is a complex mixture, bitumen-containing samples are also complex mixtures. Some chemical additives do not have sufficiently unique chemical structures that can be detected by simple colorimetric procedures or even more sophisticated methods such as gas chromatography or gas chromatography coupled to mass spectrometry because they co-elute with some bitumen components. At low ppm concentrations it is difficult, if not impossible, to quantify a specific chemical additive due to these co-elutions.

It would be desirable to have an analytical method that can measure trace level concentration of chemical additives in oil sands production fluids. Such a method to determine ppm chemical additive concentrations in process streams would enable better process control and resolution of process upsets.

### SUMMARY OF THE INVENTION

The present invention is such an analytical method for determining trace levels of a chemical additive in a production fluid from an oil recovery process, the method comprising: (a) obtaining a sample of production fluid from an oil recovery process; (b) optionally centrifuging the sample to separate suspended solids and/or to break emulsions; (c) extracting the production fluid with an organic solvent; (d) analyzing the organic solvent for the chemical additive after the extraction by multi-dimensional gas chromatography; and (e) determining the amount of chemical additive using a detector coupled to the multi-dimensional gas chromatograph.

In one embodiment of the method of the present invention described herein above, the production fluid is an oil/water mixture prior to separating the oil component from the water component.

In another embodiment of the method of the present invention described herein above, the production fluid is a water component separated from an oil/water mixture.

In another embodiment of the method of the present invention described herein above, the production fluid is from an oil sands recovery process.

In another embodiment of the method of the present invention described herein above, the extraction step utilizes a liquid-liquid extraction based on a piston-cylinder principle.

In another embodiment of the method of the present invention described herein above, the organic solvent is chloroform.

In another embodiment the method of the present invention described herein above, utilizes two capillary chromatographic columns comprising polydimethylsiloxane (PDMS), functionalized PDMS, ionic liquids, ionic sorbents, or polyethylene glycol, wherein the two columns have similar or dissimilar solute-stationary phase selectivity.

In another embodiment of the method of the present invention described herein above, the chemical additive is a glycol ether additive.

### BRIEF DESCRIPTION OF THE DRAWINGS

FIG. 1 is the two-dimensional gas chromatography chromatographic system configuration and flow profile for Example 1.

FIG. 2 is a two-dimensional gas chromatography chromatogram showing trace amounts of propylene glycol phenyl ether isolated from a bitumen process fluid.

### DETAILED DESCRIPTION OF THE INVENTION

The present invention is related to determining the level of a chemical additive in a production fluid from an oil recovery process, preferably from oil recovery processes for heavy oil and bitumen from oil sands.

Several commercial technologies are available to produce bitumen from oil sands. For shallow reservoirs, oil sands are mined then treated with warm caustic solution to separate bitumen from sand. For deeper reservoirs, Steam-Assisted Gravity Drainage (SAGD) and Cyclic Steam Stimulation (CSS) are used. In both methods, steam is injected into the reservoir to heat the formation and thereby decrease the viscosity of bitumen, which can then flow toward a production well and be pumped to the surface. In all cases, the production process yields a bitumen/water mixture that undergoes additional unit operations for bitumen/water separation and water treatment. A variety of chemicals are used at different process steps to facilitate bitumen extraction from sand, bitumen/water separation, and water treatment, for example see U.S. Pat. No. 7,938,183 and US Publication No. 20130081808. Reliable measurement of additive concentration in process streams enables better process control and resolution of process upsets. Process samples would be bitumen-containing water samples with bitumen content that could range anywhere from trace (oily water) to 99.5% (pipeline quality).

Bitumen is composed of thousands of components. Each has some degree of solubility in water, however small. Thus, the difficulty is that some additives do not have sufficiently unique chemical structures that can be detected by simple colorimetric procedures or even more sophisticated methods such as gas chromatography or gas chromatography coupled to mass spectrometry. There are so many components present that co-elution of bitumen components with the additive of interest is inevitable. At trace concentrations it is not possible to quantify the additive due to these co-elutions. This invention provides a solution to that problem by using a gas chromatograph with a primary column to separate components in time. A portion of the effluent containing the additive of interest along with co-eluting components is diverted (heart-cut) onto a second column. This second column is chosen to have a sufficiently different selectivity so as to fully separate the additive from the components that co-eluted on the first column.

Broadly, an additive is defined as a chemical having dissimilar boiling point range and polarity than constituents in the bitumen. Typical chemical additives may act as acid scavengers, cleaning agents, corrosion inhibitors, coupling agents, demulsifiers, dispersants, oxygen scavengers, hydrosulfate scavengers, surfactants, surface active agents, scale inhibitors, water clarifiers, solvents, rheology modifiers, shale inhibitors, fluid loss additives, lubricants, bridging agents, and the like. The chemical additives may be a chemical compound and/or a polymeric material. The chemical additives may be organic compounds comprising linear and/or cyclic aliphatic, aromatic moieties, or combinations. The chemical additives may comprise one or more functionality such as an ether, an amine, an ester, an alcohol, an acid, metal containing complexes, peroxides, salts, and the like.

A typical chemical additive is a surfactant or solvation aid such as an alkylene glycol ether. Preferably, the alkylene glycol ether is volatile at the temperature, pressure and environment of the steam composition when injected into a well as described above. Preferably, the alkylene glycol ether forms an azeotrope with water in order to optimize efficiency in dispersion and transport in steam. The steam composition can contain one alkylene glycol ether or a mixture of more than one kind of alkylene glycol ether.

The alkylene glycol ether is not limited in composition. Desirably, the alkylene glycol ether is selected from monoalkylene, dialkylene and trialkylene glycol ethers as opposed to polyalkylene glycol ethers having more than three alkylene glycol units. The shorter monoalkylene, dialkylene and trialkylene (especially the mono and dialkylene) glycol ethers tend to: (a) be more volatile and have better mobility with the steam; and (b) penetrate into oil sands more quickly and readily than larger polyalkylene glycol ethers.

Examples of desirable alkylene glycol ethers include those selected from a group consisting of ethylene glycol ether, propylene glycol ether and butylene glycol ether. Especially desirable are monoalkylene, dialkylene and trialkylene versions of ethylene glycol ether, propylene glycol ether and butylene glycol ether. The alkylene glycol ether can be selected from monoalkylene and dialkylene versions, or even just monoalkylene versions, of ethylene glycol ether, propylene glycol ether and butylene glycol ether. Surprisingly, the selected alkylene glycol ether can be the propylene glycol ether and/or butylene glycol ethers.

Specific examples of suitable alkylene glycol ethers include any one or any combination of more than one of the following: propylene glycol phenyl ether, dipropylene glycol monobutyl ether, propylene glycol n-butyl ether, dipropylene glycol methyl ether, dipropylene glycol n-propyl ether, propylene glycol n-propyl ether, dipropylene glycol n-butyl ether, ethylene glycol monohexyl ether, ethylene glycol mono-n-propyl ether, diethylene glycol monohexyl ether, ethylene glycol mono-n-propyl ether, diethylene glycol monobutyl ether, and triethylene glycol monobutyl ether.

After injecting the steam composition into a subterranean location containing bitumen, the bitumen recovery process further includes extracting the bitumen from the production fluid once out of the subterranean location and above the ground.

Preferred bitumen recovery processes can take the form of a cyclic steam stimulation (CSS) process where bitumen is pumped up the same well that the steam composition is injected, a steam assisted gravity drainage (SAGD) where bitumen is pumped up a second well other than the well through which the steam composition is injected into the ground, or conceivable a combination of both CSS and SAGD type processes.

The amount of alkylene glycol ether required in the steam composition to achieve improvement in bitumen extraction over steam alone is surprisingly low. The steam composition can contain as little as 0.01 weight-percent (wt %) of alkylene glycol ether and still demonstrate an improvement in bitumen extraction over use to steam alone in the same process. Typically, the steam composition contains 0.05 wt % or more, more typically 0.1 wt % or more, more typically 0.2 wt % or more, and can contain 0.3 wt % or more, 0.4 wt % or more or 0.5 wt % or more alkylene glycol ether. At the same time, the steam composition can contain 25 wt % or less, yet preferably contains 10 wt % or less, more preferably 7 wt % or less, yet more preferably 5 wt % or less and can contain 4 wt % or less alkylene glycol ether. The wt % of alkylene glycol ether is based on total combined weight of steam and alkylene glycol ether.

Excessive amounts of alkylene glycol ether cause the cost of the process to increase so lower concentrations of the alkylene glycol ether are desirable from a cost standpoint and being able to determine the level of glycol ether in the production fluid. For instance, when an additive is initially injected there is potential for some of it to be adsorbed in the formation. Monitoring the amount in the production fluid will make it possible for the operator of the well to adjust for variation in geology as the steam chambers develop underground. Also in doing trials with steam additives it is essential to monitor the fate of the additive so that there is quantitative data to base future improvements.

The method of the present invention to determine the level of a chemical additive in a production fluid from an oil recovery process comprising the steps of: (a) obtaining a sample of production fluid from an oil recovery process; (b) extracting the production fluid with an organic solvent; (c) analyzing the organic solvent for the chemical additive after the extraction by multi-dimensional gas chromatography; and (d) determining the amount of chemical additive using a detector coupled to the multi-dimensional gas chromatograph.

In one embodiment, the fluid stream is pumped out of the ground and into above ground equipment designed to separate water from bitumen. A sample can be taken anywhere in the processing equipment up to the point that the recycled water is converted back into steam, preferably from the well head. For example the production stream from a well may be diverted to a “test separator” for sampling, however, samples can be taken before the separator as well. In another embodiment, one or more test wells may be drilled into the steam chambers and samples may be obtained from these.

Samples obtained from the field can potentially have distinct oil and water phases and suspended solids. Prior to analysis, a sample of the production fluid to be analyzed may optionally be subjected to centrifugation to remove suspended solids or the distinct oil layer. The aqueous fraction may alternatively, or in addition to centrifugation, be filtered with a 0.2 μm particulate filter. An aliquot of the aqueous sample is then subjected to liquid-liquid extraction with an organic solvent. Any suitable extraction method can be used, for example a liquid-liquid extraction with a separatory funnel, with mechanical sonication, with wrist-action mechanical agitator or other suitable means can be used. A preferable method utilizes a piston-cylinder principle, for a good description see I. Peleg, S. Vromen, Chem. Ind. 15 (1983) 615 and T. Parliment, Perfume and Flavorist, 11 (1986) 4. One such piston-cylinder apparatus is called a MIXXOR™.

The MIXXOR consists of a graduated glass reservoir, a glass mixer-separator piston, provided with an axial channel, which leads into a collecting container, a screw cap, and a plastic holder-spacer. The production fluid and organic solvent to be separated are placed in the reservoir and the piston is introduced into the top of the mixing reservoir. The cap is screwed on tightly to produce a closed system and, with the holder-spaces in the upper position, the piston is pushed fully into the mixing reservoir in an up and down movement, five to six times. This forces the two liquids to pass back and forth through the narrow channel between the upper and lower containers. During this process the liquid mass is broken up into very small droplets, causing an intimate mixing of the two liquid phases. This results in a highly efficient mass transfer operation.

At the end of the mixing operation the place of the plastic space-holder is adjusted to retain the piston in the upper position, slightly above the level of the mixed liquids. The screw cap is opened slightly to release pressure. The mixed liquids are allowed to separate spontaneously into an upper (organic) and a lower (aqueous) phase. The piston is pushed carefully into the mixing reservoir, causing the upper phase to rise through the axial channel into the collecting container. The piston is stopped at the point where the lower phase reaches the top of the axial channel. The piston is kept in this position by the holder-spacer, the screw cap is removed and the liquid in the collecting container is decanted.

The incorporation of a commercially available novel piston-extraction device is advantageous as it substantially speeds up the extraction time by a factor of at least 15 times (2 min versus 30 min) when compared to mechanical agitation or sonication. Further, an extraction efficiency reaching nearly 100 percent for the target compound can be achieved. Further, the piston-extraction technology requires a minimum amount of solvent, as little as 0.5 ml per extraction, thereby saving cost of solvent as well as cost of solvent disposal with a greener chemistry approach towards sample preparation. Further, piston-extraction technology, under optimal conditions, allows for a smaller sample as compared to classical techniques. This can be advantageous where limited amount of sample is available for chemical analysis.

Any suitable organic solvent that is immiscible with water and have similar polarity may be used for the extraction step. Preferable solvents are chloroform and methylene chloride.

The organic phase comprising the chemical additive to be analyzed may be injected as is or evaporated down for sample enrichment.

In one embodiment, the chemical additive of interest may partition partially into the oil phase and partially into the water phase. In this case, an accurate determination could be achieved by a spiking study to determine the additive yield from the sample preparation steps, so that the final GC data can be translated back into the original concentration in the emulsion sample.

Multi-dimensional gas chromatography where two or more columns are connected serially or integrally formed with each other is well known, for example see U.S. Pat. Nos. 5,135,549; 7,914,612; 8,517,092; and for good reviews see J. Seeley, J. Chromatogr. A, 1255 (2012) 24 and P. Tranchida, D. Sciaronne, P. Dugo, L. Mondello, Anal. Chimica. Acta,. 716 (2012) 66. Two-dimensional gas chromatography (2-D GC) has a first and a second column connected serially with the inlet port of the second column communicating with the outlet of the first column. A sample mixture is injected in the inlet port of the first column and carried through it by a carrier gas. The sample is separated into bands as the sample is carried through the first column. One portion, or in some cases, several portions of the sample from the first column are moved by carrier gas to the second column where further chromatographic separation occurs. Separated components are detected near the outlet opening of the second column.

2-D GC separations are categorized either as heart-cutting 2-D GC (GC-GC) or as comprehensive two-dimensional gas chromatography (GC×GC). Heart-cutting 2-D GC separations pass a subset of the sample components to the secondary column and are best suited for the analysis of a few constituents. In contrast, GC×GC separations pass all sample components through both separation stages and are best suited for the complete analysis of composition. Typically, the second dimension is faster than the first dimension. The increased speed of the second dimension may be obtained by any of, or a combination of, several structural and operational differences between the first and second dimension, such as: (1) the column of the second section may have a substantially smaller diameter than the column of the first, which increases the speed of the second column through combined increases of column efficiency and carrier gas flow velocity; (2) the second column may have higher gas velocity than the first column because of the addition of carrier gas near the outlet of the first column and the inlet of the second; (3) the thickness of the stationary phase in the second column may be less than that of the first column; (4) the second column may be operated at a higher temperature than the first, or, be subjected to a different temperature program than the first; (5) the second column may have imposed upon its longitudinal axis a negative thermal gradient, which in combination with temporal temperature programming, may exert focusing effects; and (6) the stationary phase of the second column may differ in its chemical composition from that of the first.

In some embodiments, the second column has a retention time which is no more than about 25 percent the retention time of the first column and substantially all sample and a carrier gas flows through both the first and second columns.

In two-dimensional gas chromatography, the first and second columns may be two separate columns or integrally formed with each other. For separate columns, the effluent of the first column can be sent to the second column either by means of valves or by pneumatics. In using two-dimensional columns, one or more portions of sample eluting from the outlet port of the first column are diverted into the second column. Slices of eluted bands or one to several entire bands are injected into the second column where they are further separated prior to detection.

Preferred gas chromatography stationary phases are polydimethylsiloxane (PDMS), functionalized PDMS, ionic liquids, ionic sorbents, or polyethylene glycol. The two columns may have similar solute-stationary phase selectivity. Preferably, the two columns have dissimilar solute-stationary phase selectivity. Suitable capillary columns have dimensions ranging from 100 micrometers (μm) to 530 μm internal diameter. Suitable capillary columns have lengths from 1 meter to 30 meters. Suitable capillary columns have a phase thickness from 0.1 μm to 8 μm. Suitable capillary columns comprise deactivated, but uncoated fuses silica. Alternatively, packed columns with stationary phases on a support materials, for example CHROMOSORB™ W HP, can be used. Any suitable inert gases may be used as the carrier gas, for example nitrogen or more preferably hydrogen or helium.

Detection of the chemical additive may be accomplished by use of a mass spectrometer. A variety of detectors, other than mass spectrometry, can also be used for coupling with gas chromatography for sample analysis, including a flame ionization detector, a thermal conductivity detector, a pulse flame photometric detector, or an electron capture detector.

### EXAMPLE

### Example 1

Approximately 10 g of a bitumen/water sample comprising 250 ppm (w/w) of propylene glycol phenyl ether is centrifuged for phase separation. After separation, 2 ml of the filtered aqueous fraction is extracted with 0.4 ml of chloroform with a MIXXOR™ type piston extractor. The extractant as obtained is analyzed per conditions described below.

### 2D GC Analytical Conditions:

Gas chromatograph: Agilent 6890N series

Inlet: Split/splitless in split mode, inlet temperature: 200° C., split ratio 5:1

Oven profile:

60° C. (0.5 min) 15° C./min 270° C. (7 min)

### Columns:

1D: 15 m×0.25 mm id×0.1 μm DB-1HT™ wall coated fused silica column 2D: 25 m×0.25 mm id×0.25 μm VF-200™ ms wall coated fused silica column, both capillary columns available from Agilent Technologies.

### Pressure Conditions:

Inlet pressure: 24.12 psig Helium for the first dimension

Auxiliary pressure: 20.54 psig Helium for the second dimension

### Detector: Flame Ionization

Temperature: 250° C.

Hydrogen: 30 mL/min

Air: 350 mL/min

Nitrogen: 25 mL/min

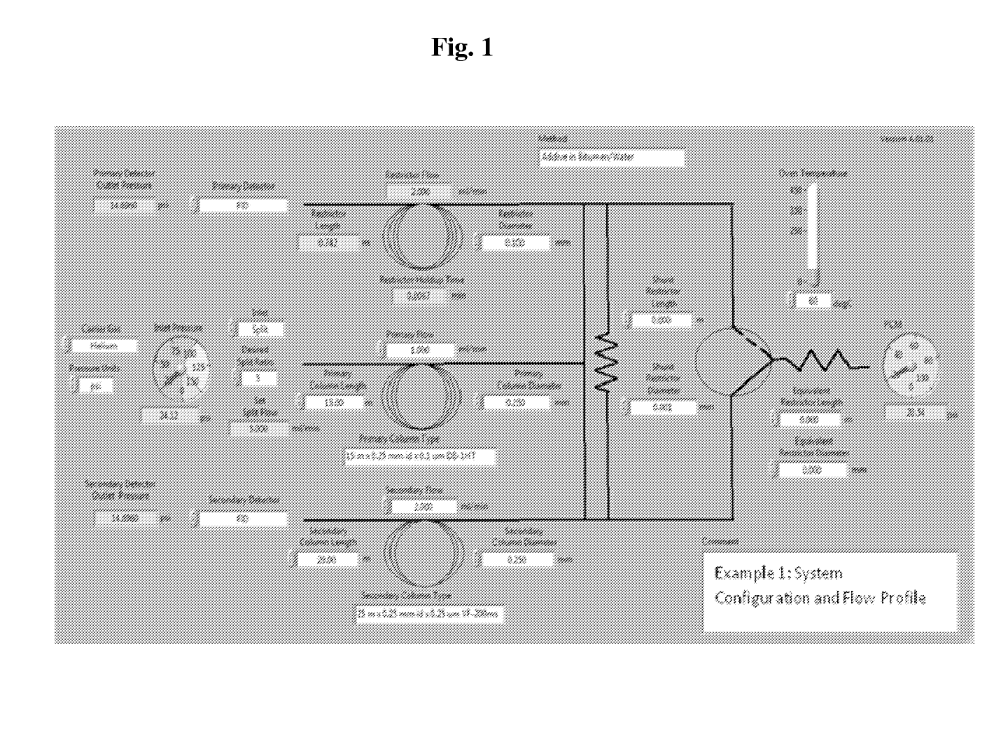

The chromatographic system configuration and flow profile is shown in FIG. 1.

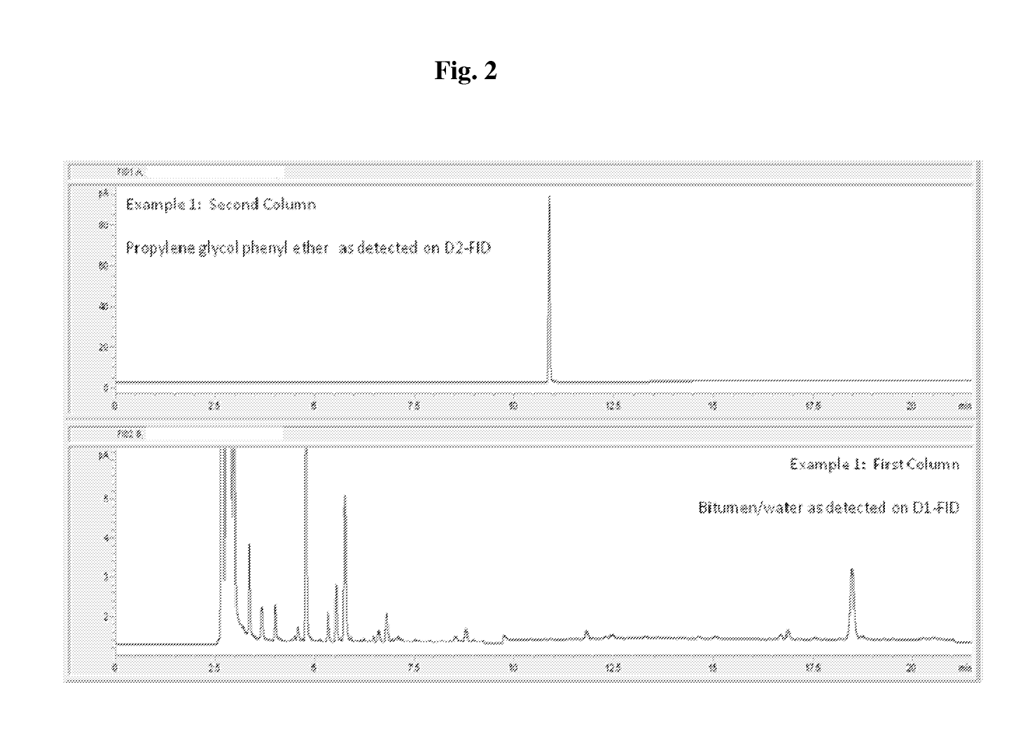

The analysis chromatogram for Example 1 is shown in FIG. 2. The chromatogram clearly shows the isolation of the propylene glycol phenyl ether in the second column and that the impurities from bitumen/water matrix are quarantined in the first column.

What is claimed is:

1. A method of determining the level of a chemical additive in a production fluid from an oil recovery process, the method comprising:
(a) obtaining a sample of production fluid from an oil recovery process;
(b) optionally centrifuging the sample to separate suspended solids and/or to break emulsions;
(c) extracting the production fluid with an organic solvent;
(d) analyzing the organic solvent for the chemical additive after the extraction by multi-dimensional gas chromatography; and
(e) determining the amount of chemical additive using a detector coupled to the multi-dimensional gas chromatograph.

2. The method of claim 1 wherein the production fluid is an oil/water mixture prior to separating the oil component from the water component.

3. The method of claim 1 wherein the production fluid is a water component separated from an oil/water mixture.

4. The method of claim 1 wherein the production fluid is from an oil sands recovery process.

5. The method of claim 1 wherein the extraction step utilizes a liquid-liquid extraction based on a piston-cylinder principle.

6. The method of claim 1 wherein the organic solvent is chloroform.

7. The method of claim 1 utilizing two capillary chromatographic columns comprising PDMS, functionalized PDMS, ionic liquids, ionic sorbents, or polyethylene glycol, wherein the two columns have similar or dissimilar solute-stationary phase selectivity.

8. The method of claim 1 wherein the chemical additive is a glycol ether additive.
